(19) United States
(12) Patent Application Publication
(10) Pub. No.: US 2003/0235600 A1
(43) Pub. Date: Dec. 25, 2003

(54) Cosmetic compositions comprising at least one polyester and at least one polyheteroatom

(75) Inventors: Balanda Atis, Newark, NJ (US); Wei Wu, Edison, NJ (US)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 10/449,150

(22) Filed: Jun. 2, 2003

Related U.S. Application Data
(60) Provisional application No. 60/384,162, filed on May 31, 2002.

(51) Int. Cl. A61K 7/00

(52) U.S. Cl. 424/401

(57) ABSTRACT

The present invention relates to a composition useful as a cosmetic containing at least one structuring polymer with a polymer skeleton having at least one hydrocarbon-based repeating unit with at least one hetero atom and at least one polyester resin.

### CROSS-REFERENCE TO A RELATED APPLICATION

The present application claims the benefit of provisional application No. 60/384,162 filed May 31, 2002.

### BACKGROUND OF THE INVENTION

Many cosmetic compositions, including pigmented cosmetics such as foundations, concealers, lipsticks, mascaras, and other cosmetic and sunscreen compositions, have been developed for longer wear and non-transfer properties. This is accomplished by the use of compositions that form a film after application. Such compositions generally contain volatile solvents which evaporate on contact with the skin or other keratinous tissue, leaving behind a layer comprising waxes and/or resins, pigments, fillers, and actives. However, these compositions tend to be uncomfortable for the wearer as the composition remains on the skin or other keratinous tissue as a brittle or non-flexible film. Such compositions may not be either supple, pliable or soft, and they may not be comfortable to wear. There may also be a tendency for such compositions to flake off because of poor adherence to the skin or other keratinous tissue. Furthermore, compositions may have a tendency to be tacky, resulting in poor application and spreadability characteristics.

Cosmetic compositions comprising at least one structuring polymer comprising a polymer skeleton which comprises at least one hydrocarbon-based repeating unit comprising at least one hetero atom have been described in WO 0152799, US 2001031280, EP 1068856, EP 1068855.

Polyester based polymers have been disclosed in sunscreen formulations in U.S. Pat. No. 5,833,961 where a functionalized polyester is used to improve the water repellency. Similar polymers have been used in U.S. Pat. No. 5,989,527 for the purpose of enhancing the performance of dermatological agents. Lipstick compositions have been disclosed in U.S. Pat. No. 6,139,823 and U.S. Pat. No. 6,074,654 where the polyester acts as a film former. A top coat lip composition containing polyester has also been described for the purpose of improving resistance in U.S. Pat. No. 6,019,962. Nail polish compositions comprising functionalized polyesters have been disclosed in U.S. Pat. No. 5,807,540. However, these compositions suffer from the lack of real comfort to the wearer as the films formed tend to be stiff and somewhat brittle.

The need therefore still remains for improved long-wearing cosmetic compositions which transfer little or not at all, i.e., “transfer-free” or transfer resistant compositions which also possess good cosmetic properties such as pliability and comfort. For example, a composition which is transfer resistant may deposit a film onto a keratinous substance which may not transfer when the keratinous substance comes into contact with, for example, skin, a cup, paper, cigarette, or a handkerchief.

It was unexpectedly discovered that the combination of at least one polyheteroatom polymer and at least one polyester resin results in a flexible, comfortable, shiny film which also exhibits transfer resistance.

### SUMMARY OF THE INVENTION

To achieve at least one of these and other advantages, the present invention, in one aspect, provides a composition comprising a liquid fatty phase comprising (i) at least one structuring polymer comprising a polymer skeleton which comprises at least one hydrocarbon-based repeating unit comprising at least one hetero atom and (ii) at least one film-forming polyester. In one embodiment, the at least one structuring polymer and the at least one film-forming polyester resin are present in an amount effective to provide transfer resistant properties, and may also provide at least one of the following properties: pliability, softness, and wearing comfort.

The present invention also relates to a method for making such a composition, for example, by combining/mixing the at least one structuring polymer and the at least one polyester resin.

Another object of the present invention is to provide methods for making-up the skin and/or hair by applying the composition of the at least one structuring polymer and the at least one polyester resin to the skin and/or hair in an amount sufficient to provide the desired effect.

### DETAILED DESCRIPTION OF THE INVENTION

One subject of the invention is cosmetic and/or dermatological compositions which are useful for the care, make-up and/or treating of at least one keratinous material which may be of suitable hardness to allow preparation of these compositions in the form of a stick or other structured form which may be stable.

The invention applies not only to make-up products for at least one keratinous material such as lip compositions, lip pencils, foundations including foundations which may be cast in the form of a stick or a dish, concealer products, temporary tattoo products, eyeliners, mascara bars but also to body hygiene products such as deodorant sticks, and to care products and products for treating at least one keratinous material such as sunscreen and after-sun products which may be in stick form. The present invention may be in the form of mascara product including mascara bars, an eyeliner product, a foundation product, a lipstick product, a blush for cheeks or eyelids, a deodorant product, a make-up product for the body, a make-up-removing product, an eyeshadow product, a face powder product, a concealer product, a treating shampoo product, a hair conditioning product, a sun screen, colorant for the skin or hair, or skin care formula such as, for example, anti-pimple or shaving cut formulas. As defined herein, a deodorant product is a body hygiene product and does not relate to care, make-up, or treatment of keratin materials, including keratin fibers, skin, or lips.

For example, the composition of the present invention may be in a form chosen from a paste, a solid, a gel, and a cream. It may be an emulsion, such as an oil-in-water or water-in-oil emulsion, a multiple emulsion, such as an oil-in-water-in-oil emulsion or a water-in-oil-in-water emulsion, or a solid, rigid or supple gel, including anhydrous gels. In one embodiment, the composition of the invention is anhydrous. The composition of the invention may, for example, comprise an external or continuous fatty phase. In another embodiment, the composition of the invention is transparent or clear, including for example, a composition without pigments. The composition can also be in a form chosen from a translucent anhydrous gel and a transparent anhydrous gel. The composition can also be a molded composition or cast as a stick or a dish. The composition in one embodiment is a solid such as a molded stick or a poured stick.

Liquid Fatty Phase

The at least one liquid fatty phase, in one embodiment, may comprise at least one oil. The at least one oil, for example, may be chosen from polar oils and apolar oils including hydrocarbon-based liquid oils and oily liquids at room temperature. In one embodiment, the compositions of the invention comprise at least one structuring polymer and at least one polar oil. The polar oils of the invention, for example, may be added to the apolar oils, the apolar oils acting in particular as co-solvent for the polar oils.

According to the invention, the structuring of the at least one liquid fatty phase may be obtained with the aid of at least one structuring polymer, such as the polymer of formula (I). In general, the polymers of formula (I) may be in the form of mixtures of polymers, these mixtures also possibly containing a synthetic product corresponding to a compound of formula (I) in which n is 0, i.e., a diester.

The liquid fatty phase of the composition may contain more than 30%, for example, more than 40%, of liquid oil(s) having a chemical nature close to the chemical nature of the skeleton (hydrocarbon or silicone based) of the structuring polymer, and for example from 50% to 100%. In one embodiment, the liquid fatty phase structured with a polyamide-type skeleton, or polyurea, or polyurethan, or polyurea-urethane-type skeleton contains a high quantity, i.e., greater than 30%, for example greater than 40% relative to the total weight of the liquid fatty phase, or from 50% to 100%, of at least one apolar, such as hydrocarbon-based, oil. For the purposes of the invention, the expression <<hydrocarbon-based oil>> means an oil comprising carbon and hydrogen atoms, optionally with at least one group chosen from hydroxyl, ester, carboxyl and ether groups.

For a liquid fatty phase structured with a polymer containing a partially silicone-based skeleton, this fatty phase may contain more than 30%, for example, more than 40%, relative to the total weight of the liquid fatty phase and, for example, from 50% to 100%, of at least one silicone-based liquid oil, relative to the total weight of the liquid fatty phase.

For a liquid fatty phase structured with an apolar polymer of the hydrocarbon-based type, this fatty phase may contain more than 30%, for example more than 40% by weight, and, as a further example, from 50% to 100% by weight, of at least one liquid apolar, such as hydrocarbon-based, oil, relative to the total weight of the liquid fatty phase.

For example, the at least one polar oil useful in the invention may be chosen from:

hydrocarbon-based plant oils with a high content of triglycerides comprising fatty acid esters of glycerol in which the fatty acids may have varied chain lengths from C₄ to C₂₄, these chains possibly being chosen from linear and branched, and saturated and unsaturated chains; these oils can be chosen from, for example, wheat germ oil, corn oil, sunflower oil, karite butter, castor oil, sweet almond oil, macadamia oil, apricot oil, soybean oil, cotton oil, alfalfa oil, poppy oil, pumpkin oil, sesame oil, marrow oil, rapeseed oil, avocado oil, hazelnut oil, grape seed oil, blackcurrant seed oil, evening primrose oil, millet oil, barley oil, quinoa oil, olive oil, rye oil, safflower oil, candlenut oil, passion flower oil and musk rose oil; or alternatively caprylic/capric acid triglycerides such as those sold by Stearineries Dubois or those sold under the names Miglyol 810, 812 and 818 by Dynamit Nobel;

synthetic oils or esters of formula R₅COOR₆ in which R₅ is chosen from linear and branched fatty acid residues containing from 1 to 40 carbon atoms and R₆ is chosen from, for example, a hydrocarbon-based chain containing from 1 to 40 carbon atoms, on condition that R₅+R₆≧10, such as, for example, purcellin oil (cetostearyl octanoate), isononyl isononanoate, C₁₂-C₁₅ alkyl benzoates, isopropyl myristate, 2-ethylhexyl palmitate, isostearyl isostearate and alkyl or polyalkyl octanoates, decanoates or ricinoleates; hydroxylated esters such as isostearyl lactate and diisostearyl malate; and pentaerythritol esters;

synthetic ethers containing from 10 to 40 carbon atoms;

C₈ to C₂₆ fatty alcohols such as oleyl alcohol; and

C₈ to C₂₆ fatty acids such as oleic acid, linolenic acid or linoleic acid.

The at least one apolar oil according to the invention is chosen from, for example, silicone oils chosen from volatile and non-volatile, linear and cyclic polydimethylsiloxanes (PDMSs) that are liquid at room temperature; polydimethylsiloxanes comprising alkyl or alkoxy groups which are pendant and/or at the end of the silicone chain, the groups each containing from 2 to 24 carbon atoms; phenylsilicones such as phenyl trimethicones, phenyl dimethicones, phenyl trimethylsiloxy diphenylsiloxanes, diphenyl dimethicones, diphenyl methyldiphenyl trisiloxanes and 2-phenylethyl trimethylsiloxysilicates; hydrocarbons chosen from linear and branched, volatile and non-volatile hydrocarbons of synthetic and mineral origin, such as volatile liquid paraffins (such as isoparaffins and isododecane) or non-volatile liquid paraffins and derivatives thereof, liquid petrolatum, liquid lanolin, polydecenes, hydrogenated polyisobutene such as Parleam®, and squalane; and mixtures thereof. The structured oils, for example those structured with polyamines such as those of formula (I) or with polyurethanes, polyureas, polyurea-urethanes, in accordance with the invention, may be, in one embodiment, apolar oils, such as an oil or a mixture of hydrocarbon oils chosen from those of mineral and synthetic origin, chosen from hydrocarbons such as alkanes such as Parleam® oil, isoparaffins including isododecane, and squalane, and mixtures thereof. These oils may, in one embodiment, be combined with at least one phenylsilicone oil.

The liquid fatty phase, in one embodiment, contains at least one non-volatile oil chosen from, for example, hydrocarbon-based oils of mineral, plant and synthetic origin, synthetic esters or ethers, silicone oils and mixtures thereof.

In practice, the total liquid fatty phase can be, for example, present in an amount ranging from 1% to 99% by weight relative to the total weight of the composition, for example from 5% to 95.5%, from 10% to 80%, or from 20% to 75%.

For the purposes of the invention, the expression “volatile solvent or oil” means any non-aqueous medium capable of evaporating on contact with the skin or the lips in less than one hour at room temperature and atmospheric pressure. The volatile solvent(s) of the invention is (are) organic solvents, such as volatile cosmetic oils that are liquid at room temperature, having a non-zero vapor pressure, at room temperature and atmospheric pressure, ranging in particular from 10⁻² to 300 mmHg and, for example, greater than 0.3 mmHg. The expression <<non-volatile oil>> means an oil which remains on the skin or the lips at room temperature and atmospheric pressure for at least several hours, such as those having a vapor pressure of less than 10⁻² mmHg.

According to the invention, these volatile solvents may facilitate the staying power or long wearing properties of the composition on the skin, the lips or superficial body growths, such as keratinous fibers. The solvents can be chosen from hydrocarbon-based solvents, silicone solvents optionally comprising alkyl or alkoxy groups that are pendant or at the end of a silicone chain, and a mixture of these solvents.

The volatile oil(s), in one embodiment, is present in an amount ranging up to 95.5% relative to the total weight of the composition, such as from 2% to 75%, and, as a further example, from 10% to 45%. This amount will be adapted by a person skilled in the art according to the desired staying power or long wearing properties.

The at least one liquid fatty phase of the compositions of the invention may further comprise a dispersion of lipid vesicles. The compositions of the invention may also, for example, be in the form of a fluid anhydrous gel, a rigid anhydrous gel, a fluid simple emulsion, a fluid multiple emulsion, a rigid simple emulsion or a rigid multiple emulsion. The simple emulsion or multiple emulsion may comprise a continuous phase chosen from an aqueous phase optionally containing dispersed lipid vesicles or oil droplets, or a fatty phase optionally containing dispersed lipid vesicles or water droplets. In one embodiment, the composition has a continuous oily phase or fatty phase and is more specifically an anhydrous composition, for example, a stick or dish form. An anhydrous composition is one that has less than 10% water by weight, such as, for example, less than 5% by weight.

Structuring Polymer

In one embodiment, the at least one structuring polymer in the composition of the invention is a solid that is not deformable at room temperature (25° C.) and atmospheric pressure (760 mmHg). In a further embodiment, the at least one structuring polymer is capable of structuring the composition without opacifying it. As defined above, the at least one structuring polymer of the present invention comprises a polymer skeleton comprising at least one hydrocarbon-based repeating unit comprising at least one hetero atom. In one embodiment, the at least one structuring polymer further comprises at least one terminal fatty chain chosen from alkyl and alkenyl chains, such as of at least 4 atoms, and further such as comprising 8 to 120 carbon atoms, bonded to the polymer skeleton via at least one linking group. The terminal fatty chain may, for example, be functionalized. The at least one structuring polymer may also further comprise at least one pendant fatty chain chosen from alkyl and alkenyl chains, such as of at least 4 atoms, and further such as comprising 8 to 120 carbon atoms, bonded to any carbon or hetero atom of the polymer skeleton via at least one linking group. The pendant fatty chain may, for example, be functionalized. The at least one structuring polymer may comprise both at least one pendant fatty chain and at least one terminal fatty chain as defined above, and one or both types of chains can be functionalized.

In one embodiment, the structuring polymer comprises at least two hydrocarbon-based repeating units. As a further example, the structuring polymer comprises at least three hydrocarbon-based repeating units and as an even further example, the at least three repeating units are identical.

As used herein, “functionalized” means comprising at least one functional group. Non-limiting examples of functional groups include hydroxyl groups, ether groups, oxyalkylene groups, polyoxyalkylene groups, carboxylic acid groups, amine groups, amide groups, halogen containing groups, including fluoro and perfluoro groups, halogen atoms, ester groups, siloxane groups and polysiloxane groups.

For purposes of the invention, the expression “functionalized chain” means, for example, an alkyl chain comprising at least one functional (reactive) group chosen, for example, from those recited above. For example, in one embodiment, the hydrogen atoms of at least one alkyl chain may be substituted at least partially with fluorine atoms.

According to the invention, these chains may be linked directly to the polymer skeleton or via an ester function or a perfluoro group.

For the purposes of the invention, the term “polymer” means a compound containing at least 2 repeating units, such as, for example, a compound containing at least 3 repeating units, which may be identical.

As used herein, the expression “hydrocarbon-based repeating unit” includes a repeating unit comprising from 2 to 80 carbon atoms, such as, for example, from 2 to 60 carbon atoms. The at least one hydrocarbon-based repeating unit may also comprise oxygen atoms. The hydrocarbon-based repeating unit may be chosen from saturated and unsaturated hydrocarbon-based repeating units which in turn may be chosen from linear hydrocarbon-based repeating units, branched hydrocarbon-based repeating units and cyclic hydrocarbon-based repeating units. The at least one hydrocarbon-based repeating unit may comprise, for example, at least one hetero atom that is part of the polymer skeleton, i.e., not pendant. The at least one hetero atom may be chosen, for example, from nitrogen, sulphur, and phosphorus. For example, the at least one hetero atom may be a nitrogen atom, such as a non-pendant nitrogen atom. In another embodiment, the at least one hydrocarbon-based repeating unit may comprise at least one hetero atom with the proviso that the at least one hetero atom is not nitrogen. In another embodiment, the at least one hetero atom is combined with at least one atom chosen from oxygen and carbon to form a hetero atom group. In one embodiment, the hetero atom group comprises a carbonyl group.

The at least one repeating unit comprising at least one hetero atom may be chosen, for example, from amide groups, carbamate groups, and urea groups. In one embodiment, the at least one repeating unit comprises amide groups forming a polyamide skeleton. In another embodiment, the at least one repeating unit comprises carbamate groups and/or urea groups forming a polyurethane skeleton, a polyurea skeleton and/or a polyurethane-polyurea skeleton. The pendant chains, for example, can be linked directly to at least one of the hetero atoms of the polymer skeleton. In yet another embodiment, the at least one hydrocarbon-based repeating unit may comprise at least one hetero atom group with the proviso that the at least one hetero atom group is not an amide group. In one embodiment, the polymer skeleton comprises at least one repeating unit chosen from silicone units and oxyalkylene units, the at least one repeating unit being between the hydrocarbon-based repeating units.

In one embodiment, the compositions of the invention comprise at least one structuring polymer with nitrogen atoms, such as amide, urea, or carbamate units, such as amide units, and at least one polar oil.

In one embodiment, in the at least one structuring polymer, the percentage of the total number of fatty chains ranges from 40% to 98% relative to the total number of repeating units and fatty chains, and as a further example, from 50% to 95%. In a further embodiment wherein the polymer skeleton is a polyamide skeleton, in the at least one structuring polymer, the percentage of the total number of fatty chains ranges from 40% to 98% relative to the total number of all amide units and fatty chains, and as a further example, from 50% to 95%.

In a further embodiment, the nature and proportion of the at least one hydrocarbon-based repeating unit comprising at least one hetero atom depends on the nature of a liquid fatty phase of the composition and is, for example, similar to the nature of the fatty phase. For example, not to be limited as to theory, the more polar the hydrocarbon-based repeating units containing a hetero atom, and in high proportion, which corresponds to the presence of several hetero atoms, the greater the affinity of the at least one structuring polymer to polar oils. Conversely, the more non-polar, or even apolar, and lesser in proportion the hydrocarbon-based repeating units containing a hetero atom, the greater the affinity of the polymer for apolar oils.

In another embodiment, the invention is drawn to a structured composition containing at least one liquid fatty phase structured with at least one structuring polymer, wherein the at least one structuring polymer is a polyamide comprising a polymer skeleton comprising at least one amide repeating unit and optionally at least one pendant fatty chain and/or at least one terminal chain that are optionally functionalized and comprise from 8 to 120 carbon atoms, bonded to at least one of the amide repeating units via at least one linking group.

When the structuring polymer has amide repeating units, the pendant fatty chains may be linked to at least one of the nitrogen atoms in the amide repeating units.

The structuring polymer, for example the polyamide polymer, may have a weight-average molecular mass of less than 100,000, such as less than 50,000. In another embodiment, the weight-average molecular mass may range from 1000 to 30,000, such as from 2000 to 20,000, further such as from 2000 to 10,000.

As discussed, the at least one structuring polymer may, for example, be chosen from polyamide polymers. A polyamide polymer may comprise, for example, a polymer skeleton which comprises at least one amide repeating unit, i.e., a polyamide skeleton. In one embodiment, the polyamide skeleton may further comprise at least one terminal fatty chain chosen from alkyl chains, for example, alkyl chains comprising at least four carbon atoms, and alkenyl chains, for example, alkenyl chains comprising at least four carbon atoms, bonded to the at least one polyamide skeleton via at least one linking group, and/or at least one pendant fatty chain chosen from alkyl chains, for example, alkyl chains comprising at least four carbon atoms, and alkenyl chains, for example, alkenyl chains comprising at least four carbon atoms, bonded to the at least one polyamide skeleton via at least one linking group. In one embodiment, the polyamide skeleton may comprise at least one terminal fatty chain chosen from fatty chains comprising 8 to 120 carbon atoms, such as, for example, 12 to 68 carbon atoms, bonded to the at least one polyamide skeleton via at least one linking group and/or at least one pendant fatty chain chosen chosen from fatty chains comprising 8 to 120 carbon atoms, such as, for example, 12 to 68 carbon atoms, bonded to the at least one polyamide skeleton via at least one linking group, such as bonded to any carbon or nitrogen of the polyamide skeleton via the at least one linking group. In one embodiment, the at least one linking group is chosen from single bonds and urea, urethane, thiourea, thiourethane, thioether, thioester, ester, ether and amine groups. For example, the at least one linking group is chosen from ureas, esters, and amines, and as a further example, is chosen from esters and amines. The bond is, for example, an ester bond. In one embodiment, these polymers comprise a fatty chain at each end of the polymer skeleton, such as the polyamide skeleton.

In one embodiment, due to the presence of at least one chain, the polyamide polymers may be readily soluble in oils (i.e., water-immiscible liquid compounds) and thus may give macroscopically homogeneous compositions even with a high content (at least 25%) of the polyamide polymers, unlike certain polymers of the prior art that do not contain such alkyl or alkenyl chains at the end of the polyamide skeleton. As defined herein, a composition is soluble if it has a solubility of greater than 0.01 g per 100 ml of solution at 25° C.

In a further embodiment, the polyamide polymers can be chosen from polymers resulting from at least one polycondensation reaction between at least one acid chosen from dicarboxylic acids comprising at least 32 carbon atoms, such as 32 to 44 carbon atoms, and at least one amine chosen from diamines comprising at least 2 carbon atoms, such as from 2 to 36 carbon atoms, and triamines comprising at least 2 carbon atoms, such as from 2 to 36 carbon atoms. The dicarboxylic acids can, for example, be chosen from dimers of at least one fatty acid comprising at least 16 carbon atoms, such as oleic acid, linoleic acid and linolenic acid. The at least one amine can, for example, be chosen from diamines, such as ethylenediamine, hexylenediamine, hexamethylenediamine, phenylenediamine and triamines, such as ethylenetriamine.

The polyamide polymers may also be chosen from polymers comprising at least one terminal carboxylic acid group. The at least one terminal carboxylic acid group can, for example, be esterified with at least one alcohol chosen from monoalcohols comprising at least 4 carbon atoms. For example, the at least one alcohol can be chosen from monoalcohols comprising from 10 to 36 carbon atoms. In a further embodiment, the monoalcohols can comprise from 12 to 24 carbon atoms, such as from 16 to 24 carbon atoms, and for example 18 carbon atoms.

In one embodiment, the at least one polyamide polymer may be chosen from those described in U.S. Pat. No. 5,783,657, the disclosure of which is incorporated herein by reference, which are polymers of formula (I):

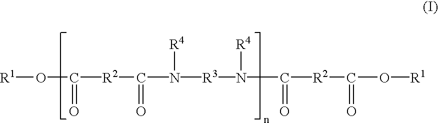

in which:

n is an integer which represents the number of amide units such that the number of ester groups present in said at least one polyamide polymer ranges from 10% to 50% of the total number of all said ester groups and all said amide groups comprised in said at least one polyamide polymer;

R¹, which are identical or different, are each chosen from alkyl groups comprising at least 4 carbon atoms and alkenyl groups comprising at least 4 carbon atoms. In one embodiment, the alkyl group comprises from 4 to 24 carbon atoms and the alkenyl group comprises from 4 to 24 carbon atoms;

R², which are identical or different, are each chosen from C₄ to C₄₂ hydrocarbon-based groups with the proviso that at least 50% of all R² are chosen from C₃₀ to C₄₂ hydrocarbon-based groups;

R³, which are identical or different, are each chosen from organic groups comprising atoms chosen from carbon atoms, hydrogen atoms, oxygen atoms and nitrogen atoms with the proviso that R³ comprises at least 2 carbon atoms; and

R⁴, which are identical or different, are each chosen from hydrogen atoms, C₁ to C₁₀ alkyl groups and a direct bond to at least one group chosen from R³ and another R⁴ such that when said at least one group is chosen from another R⁴, the nitrogen atom to which both R³ and R⁴ are bonded forms part of a heterocyclic structure defined in part by R⁴—N—R³, with the proviso that at least 50% of all R⁴ are chosen from hydrogen atoms.

In one embodiment, the at least one terminal fatty chain of formula (I) is linked to the last hetero atom, in this case nitrogen, of the polyamide skeleton. In a further embodiment, the terminal chains are functionalized. In another embodiment, the ester groups of formula (I), are linked to the terminal and/or pendant fatty chains, represent from 15% to 40% of the total number of ester and amide groups, such as, for example, from 20% to 35%.

In one embodiment, n may be an integer ranging from 1 to 5, for example, an integer ranging from 3 to 5. In the present invention, R¹, which are identical or different, can, for example, each be chosen from C₁₂ to C₂₂ alkyl groups, such as from C₁₆ to C₂₂ alkyl groups.

In the present invention, R², which are identical or different, can, for example, each be chosen from C₁₀ to C₄₂ alkyl groups. At least 50% of all R², which are identical or different, can, for example, each be chosen from groups comprising from 30 to 42 carbon atoms. At least 75% of all R² which are identical or different, can, for example, each be chosen from groups comprising from 30 to 42 carbon atoms. In the two aforementioned embodiments, the remaining R², which are identical or different, can, for example, each be chosen from C₄ to C₁₉ groups, such as C₄ to C₁₂ groups.

R³, which can be identical or different, can, for example, each be chosen from C₂ to C₃₆ hydrocarbon-based groups and polyoxyalkylene groups. In another example, R³, which can be identical or different, can each, for example, be chosen from C₂ to C₁₂ hydrocarbon-based groups. In another embodiment, R⁴, which can be identical or different, can each be chosen from hydrogen atoms. As used herein, hydrocarbon-based groups may be chosen from linear, cyclic and branched, and saturated and unsaturated groups. The hydrocarbon-based groups can be chosen from aliphatic and aromatic groups. In one example, the hydrocarbon-based groups are chosen from aliphatic groups. The alkyl and alkylene groups may be chosen from linear, cyclic and branched, and saturated and unsaturated groups.

In general, the pendant and terminal fatty chains may be chosen from linear, cyclic and branched, and saturated and unsaturated groups. The pendant and terminal fatty chains can be chosen from aliphatic and aromatic groups. In one example, the pendant and terminal fatty chains are chosen from aliphatic groups.

According to the invention, the structuring of the liquid fatty phase is obtained with the aid of at least one structuring polymer, such as the at least one polymer of formula (I). The at least one polyamide polymer of formula (I) may, for example, be in the form of a mixture of polymers, and this mixture may also comprise a compound of formula (I) wherein n is equal to zero, i.e., a diester.

Non-limiting examples of at least one polyamide polymer which may be used in the composition according to the present invention include the commercial products sold by Arizona Chemical under the names Uniclear 80 and Uniclear 100. These are sold, respectively, in the form of an 80% (in terms of active material) gel in a mineral oil and a 100% (in terms of active material) gel. These polymers have a softening point ranging from 88° C. to 94° C., and may be mixtures of copolymers derived from monomers of (i) C₃₆ diacids and (ii) ethylenediamine, and have a weight-average molecular mass of about 6000. Terminal ester groups result from esterification of the remaining acid end groups with at least one alcohol chosen from cetyl alcohol and stearyl alcohol. A mixture of cetyl and stearyl alcohols is sometimes called cetylstearyl alcohol.

Other non-limiting examples of at least one polyamide polymer which may be used in the composition according to the present invention include polyamide polymers resulting from the condensation of at least one aliphatic dicarboxylic acid and at least one diamine, the carbonyl and amine groups being condensed via an amide bond. Examples of these polyamide polymers are those sold under the brand name Versamid by the companies General Mills Inc. and Henkel Corp. (Versamid 930, 744 or 1655) or by the company Olin Mathieson Chemical Corp. under the brand name Onamid, in particular Onamid S or C. These resins have a weight-average molecular mass ranging from 6000 to 9000. For further information regarding these polyamides, reference may be made to U.S. Pat. Nos. 3,645,705 and 3,148,125, the disclosures of which are hereby incorporated by reference.

Other examples of polyamides include those sold by the company Arizona Chemical under the references Uni-Rez (2658, 2931, 2970, 2621, 2613, 2624, 2665, 1554, 2623 and 2662) and the product sold under the reference Macromelt 6212 by the company Henkel. For further information regarding these polyamides, reference may be made to U.S. Pat. No. 5,500,209, the disclosure of which is hereby incorporated by reference. Such polyamides display high melt viscosity characteristics. MACROMELT 6212, for example, has a high melt viscosity at 190° C. of 30-40 poise (as measured by a Brookfield Viscometer, Model RVF #3 spindle, 20 RPM).

In a further embodiment, the at least one polyamide polymer may be chosen from polyamide resins from vegetable sources. Polyamide resins from vegetable sources may be chosen from, for example, the polyamide resins of U.S. Pat. Nos. 5,783,657 and 5,998,570, the disclosures of which are herein incorporated by reference.

In one embodiment, the at least one polyamide polymer may be present in the composition in an amount ranging, for example, from 0.5% to 80%, such as from 2% to 60%, further such as from 5% to 40%, by weight relative to the total weight of the composition. In a further embodiment the at least one polyamide polymer may be present in the composition in an amount ranging, for example, from 5% to 25% by weight relative to the total weight of the composition.

In one embodiment, the at least one structuring polymer in the composition according to the invention corresponds to the polyamide polymers of formula (I). Due to fatty chain(s), these polymers may be readily soluble in oils and thus lead to compositions that are macroscopically homogeneous even with a high content (at least 25%) of at least one structuring polymer.

The at least one structuring polymer may have a softening point greater than 50° C., such as from 65° C. to 190° C., and further such as from 70° C. to 130° C., and even further such as from 80° C. to 105° C.

It is to be understood that both the foregoing general description and the following detailed description are exemplary and explanatory only, and are not restrictive of the invention.

Polyester Film Former

The polyesters suitable for use in the compositions are those which include a backbone derived from the reaction of at least one diol and at least one diacid. The diol is preferably a linear or branched aliphatic dihydric compound having two —OH groups. The diacid is preferably a linear or branched chain aliphatic dicarboxylic acid having two —COOH groups, although aromatic acids may also be used. The polyester backbone is preferably derived from the co-condensation of such diols and diacids. The diol may contain from 2 to 10 ether linkages (—R—O—R—) or from 2 to 10 tertiary amine groups (NR₃). The polyester may be linear or cross-linked as described below.

While the resulting polyesters generally contain —OH and/or —COOH functionality, they may be —OH end-functionalized, or preferably, the polyester is further functionalized and/or cross-linked by reacting the at least one diol and at least one diacid as noted above with at least one polyfunctional acid or polyfunctional alcohol, preferably having a functionality of at least 3 with respect to the —OH or —COOH groups. While use of bifunctional reactants may produce polyesters having functionalities of less than 2, preferably, the polyfunctional acid or polyfunctional alcohol should be provided in an amount sufficient to provide a polyester which has an enhanced functionality, either —OH or —COOH, of at least 2 and a molecular weight of preferably from 500 to 100,000. While the use of a polyfunctional acid or polyfunctional alcohol is preferred for increasing the functionality of the polyester, other suitable methods may be used provided the resulting polyester has an acceptable molecular weight and achieves an equivalent level of functionality. Preferably, if a polyfunctional acid or polyfunctional alcohol is used, it is provided in an amount of up to about 50 percent by weight of the other reactants. The resulting polyesters are either hydroxy or acid functional and preferably have either —OH or —COOH terminal end groups which thereby provides a functionalized polyester having a functionality of at least 2. A greater degree of functionality, in terms of functional groups per unit mass, is obtained if the molecular weight of the polyester is lower. Crosslinking provides an increase in cross-link density as well as contributes to increasing functionality.

The amount of acid or alcohol to be used, in equivalent weight, is determined in accordance with the number of —OH or —COOH groups, or equivalents, to be provided per unit mass of polymer. The molecular weight of the acid or alcohol can then be used to calculate the appropriate weight percentage of acid or alcohol required to achieve the desired number of equivalents. Preferably, one or less equivalent of acid or alcohol is provided per equivalent desired in the final polymer such that the resulting polymer will have substantially no free acid or alcohol remaining and all of the acid or alcohol will be used in the functionalizing reaction. As such, the equivalents and moles of acid and alcohol are selected to provide the desired molecular weight for the polyester and the desired hydroxyl and/or acid content per unit mass in the polyester.

The diacids used for forming the polyesters are preferably of from 2 to 20 carbon atoms. Suitable aliphatic diacids for use in forming the polyesters include, for example, malonic acid, maleic acid, fumaric acid, acetylene dicarboxylic acid, succinic acid, glutaric acid, adipic acid, pentanedioic acid, muconic acid, pimelic acid, suberic acid, azelaic acid, sebacic acid, undecandioic acid, traumatic acid and branched and linear alkyl, alkenyl and nonaromatic cycloalkyl and nonaromatic cycloalkenyl derivatives of those acids. Preferably, the acid is adipic acid or a branched alkyl derivative of an aliphatic acid. Preferred linear aliphatic diacids are adipic acid, glutaric acid, succinic acid, azelaic acid, and sebacic acid. Preferred branched diacids are hydrogenated and unhydrogenated dimer acids. Cyclic aliphatic acids are also useful with a preferred cyclic acid being cyclohexanedicarboxylic acid. Aromatic acids may also be used, such as orthophthalic acid, isophthalic acid and terephthalic acid. One skilled in the art, based on this disclosure, would be aware that such a list of possible diacids is exemplary in nature and that other suitable diacids having similar properties are within the scope of this disclosure.

Suitable diols for use in forming the polyesters include any alkane diol, alkene diol, alkyne diol, nonaromatic cycloalkane diol or nonaromatic cycloalkene diol and their derivatives. Preferably, such diols have from 2 to 20 carbon atoms. Such diols include, for example, ethylene glycol, propylene glycol, 1,2 butanediol, 1,5-pentanediol, cyclopentanediol, 3,3-dimethyl-1,2-butanediol, 3-hexyne-2,5-diol and the like. These diols are exemplary in nature and it should be understood by one skilled in the art, based on this disclosure, that the position of the —OH group on such diols may be varied. For example, 1,2 pentanediol, 1,3 pentanediol and 1,5 pentanediol are all suitable diols. Further, ether-containing diols having the formula HO—(CH₂—CH₂—O)_{b}—CH₂—CH₂—OH, where b is from 1 to 5, may also be used. An example of a preferred ether group-containing diol for forming the polyester of the present invention is diethylene glycol. Examples of preferred branched chain diols for use in the invention include neopentyl diol, trimethylpentane diol and similar diols.

The above diacids and diols may be used singly or in a mixture of diacids and diols. Further, as noted above, when forming the polyesters of the present invention, a polyfunctional acid or polyfunctional alcohol may be added for crosslinking the polyester and/or providing an enhanced level of —-OH or —COOH functionality. Suitable polyfunctional acids include any polycarboxylic acid having the preferred functionality as noted above. Suitable polyfunctional alcohols include functionalized alcohols having the required —OH functionality as noted above.

Preferably, a polyfunctional alcohol is used to provide crosslinking and/or additional —OH functionality to the polyester. Examples of suitable polyfunctional alcohols include 1,2,3-propanetriol (glycerin), 1,2,4-butanetriol, 1,1,1,-tris(hydroxymethyl)ethane, 1,2,6-trihydroxyhexane and the like. It will be understood from this disclosure that other polyfunctional alcohols or acids may be used to provide the required functionality and the above examples are not intended to be limiting.

The polyesters may be formed according to any suitable polymerization method. Preferably, the polyesters are formed by co-condensation of the diacids and diols in the presence of the functionalizing and/or crosslinking polyfunctional acid or polyfunctional alcohol.

In forming suitable polyesters for use in the present invention, the terminal end groups can be left as is and used in preferred topical compositions according to the present invention, or further end-functionalized by reacting with at least one compound, such as, for example, a linear or branched aliphatic monofunctional acid of from about 2 to 40 carbon atoms, a linear or branched monofunctional alcohol of from about 2 to 40 carbon atoms or combinations of such acids and alcohols. The polyesters can also be further end-functionalized by reacting them with primary or secondary amines or silicon-containing compounds. The amount of the functionalizing reactants provided are determined in accordance with the desired end-functionality. The number of equivalents per unit mass required to provide the specific functionality for a given application of the polyester is determined as described above. Based on the number of necessary equivalents per unit mass, the molecular weight of the functionalizing reactant is then used to determine the amount of that reactant to be provided to the reaction to achieve the specific functionality in the resulting polyester. Preferably, the equivalents are determined such that substantially no functionalizing reactant remains in a free form in the resulting polyester and substantially all of the reactant is used in the functionalizing reaction.

The linear and branched aliphatic monofunctional acids may include any carboxylic acid having from 2 to 40 carbon atoms. Examples of such acids include, formic acid, acetic acid, propionic acid, butyric acid, valeric acid, caproic acid, caprylic acid, capric acid, lauric acid, myristic acid, palmitic acid, stearic acid and similar linear aliphatic acids. The monofunctional group may be attached at any carbon site along the chain. Alkenyl, alkynyl, branched chain and nonaromatic cyclic acid derivatives of such linear acids having the appropriate monofunctionality may also be used, for example, 1-methylhexanoic acid or 4-hexenoic acid.

The linear and branched aliphatic monofunctional alcohols which may be used should be from about 2 to about 40 carbon atoms. Examples of such alcohols include methanol, ethanol, propanol, butanol, pentanol, hexanol and the like. The monofunctional hydroxyl group may be attached at any carbon site along the chain. Alkenyl, alkynyl, branched chain and nonaromatic cyclic alcohol derivatives of such linear alcohols having the appropriate monofunctionality may also be used, for example, propenol and similar alkenyl alcohols, isooctanol, 2-ethylhexanol, isodecanol, tridecanol and similar branched alcohols.

Exemplary cross-linked or linear polyesters for use in the present invention may be of Formula II below:

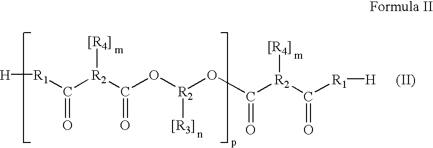

wherein:

R₁ is independently either —O— or —O—R₂—;

R₂ is independently a linear or branched C₂-C₆₀ aliphatic group;

R₃ is —O(C═O)—R₁—H, or —O—[(C═O)—R₂(R₄)ₘ—(C═O)—O—R₂(R₃)ₙ—O]ₚ(C═O)R₂(R₄)ₘ—(C═O)—R₁—H;

R₄ is —(C═O) —O—R₁—H, H, or —(C═O)—O—R₂—O—[(C═O)—R₂(R₄)ₘ—(C═O)—O—R₂(R₃)ₙ—O]ₚ{C═O)—R₂(R₄)ₘ—(C═O)—R₁—H;

m is independently an integer and 1<=m<=5;

n is independently an integer and 1<=n<=5; and

p is independently an integer and 3<=p<=2000.

Further examples of most preferred polyesters for use in the present invention include linear, acid-functional polyester polyols; hydroxy-functional polyester diols; linear, carboxylic acid-end-fucntionalized complex polyesters; linear, alcohol-end-functionalized complex polyesters; and the polyfunctional acid or polyfunctional alcohol cross-linked derivatives of these polyesters. Such preferred polyesters are shown below in Formulae III-X:

Linear, Acid-Functional Polyester Polyol:

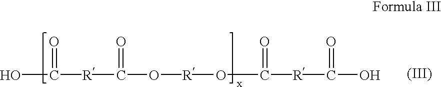

wherein R′ is independently a linear or branched hydroxy-substituted or unsubstituted aliphatic C₂-C₆₀ group with from 0 to 200 ether linkages, a hydroxy-substituted or unsubstituted cyclic aliphatic C₂-C₁₂ group, or a hydroxy-substituted or unsubstituted aromatic group; and

x is independently an integer and 3<=x<=2000.

Acid-Functional Polyester Polyol Cross-Linked With Polyfunctional Acid:

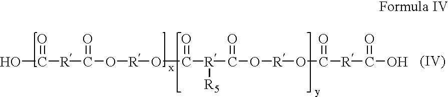

wherein R′ and x are as defined above, R₅ is —H, —(C═O)—OH or —(C═O)—R′—R₅)_{q}—(C═O)—O—R′—O]_{y}—(C═O)—R′(R₅)_{q}—(C═O)—OH;

y is independently an integer and 3<=y<=2000; and

q is independently an integer and 0<=q<=5.

Linear, Hydroxy-Functional Polyester Diol:

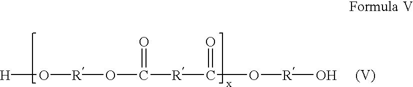

wherein R′ and x are as defined above.

Hydroxy-Functional Polyester Polyol Cross-Linked with Polyfunctional Alcohol:

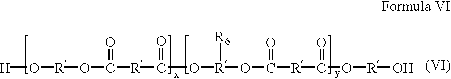

wherein x, y, q and R′ are as defined above; and R₆ is —H, OH or —[O—R′(R₆)_{q}—O—(C═O)—R′—(C═O)]_{y}—O—R′(R₆)_{q}—OH

Linear, Carboxylic Acid-End-Functionalized Complex Polyester:

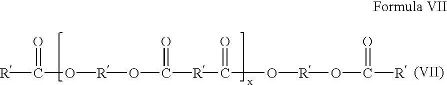

wherein R′ and x are as defined above.

Carboxylic Acid-End Functionalized Complex Polyester Cross-Linked with Polyfunctional Acid:

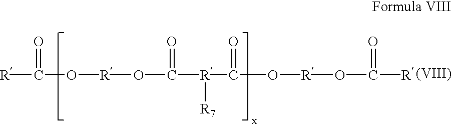

wherein R′, x and q are as defined above; and

R₇ is H, —(C═O)—OH or —[O—R′—O—(C═O)—R′(R₇)_{q}—(C═O)]ₓ—O—R′—O—C═O)]ₓ—O—R′—O—(C═O)—R′(R₇)_{q}

Linear, Alcohol-End-Functionalized Complex Polyester:

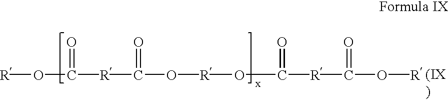

wherein R′ and x are as defined above.

Alcohol-End-Functionalized Complex Polyester Cross-Linked With Polyfunctional Alcohol:

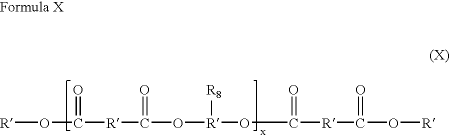

wherein R′ and x are as defined above; and

R₈ is H, —OH, or —[(C═O)—R′—(C═O)—O—R′(R₈)_{q}—O—]ₓ—(C═O)—R′—(C═O)—O—R′(R₈)

It should be understood, based on this disclosure, that while polyesters in accordance with the Formulae I-IX are preferred, other polyesters, such as those having mixed acid or alcohol-end functionalities or mixed polyfunctional alcohol and polyfunctional acid cross-linking may be used in the invention. In addition, any other suitable polyesters formed from co-condensation of compounds such as those described above which provide the desired effects for the specific active ingredients as noted herein may be used in the compositions of the present invention. However, the preferred polyesters for use in the compositions according to the present invention have solubility parameters and molecular weights within a preferred range which will enable the polyester to function best in the topical compositions as described further below. Further, more than one polyester may be used in the compositions if the effect can be modified or otherwise enhanced to provide the ability to control the rate or degree of penetration of the active ingredient into the stratum corneum once the composition has been topically applied.

In addition to the foregoing, when using the monofunctional acids and alcohols to end-functionalize the polyesters, the monofunctional acids and alcohols may incorporate tertiary amine groups within the acid or alcohol chain.

Suitable primary and secondary amines which can be used for end-functionalizing the polyesters of the present invention include amines such as XNH₂ and X₂NH where the X group is a branched or linear nonaromatic alkyl, alkenyl or alkynyl chain of from 2 to 40 carbon atoms which may be further amine substituted. The reaction of such a primary or secondary amine with polyesters of the present invention produces an amine functionalized polyester, for example, having —NHX or —NX₂ end groups, where X is a described above in place of the —H or —R′ on either end of the above Formulae. Exemplary primary and secondary amines include dimethylaminopropylamine, diethylaminoethylamine, aminoethylethanolamine, monoethanolamine, diethanolamine, isopropanolamine, commercially available saturated normal amines having from 6 to 24 carbon atoms and saturated secondary amines.

Exemplary silicon-containing compounds include polysilicones and/or polysiloxanes. Preferred polysiloxanes have an —Si(Y)₂O— repeating unit where Y is a lower alkyl group of from 1-4 carbon atoms. Hydroxy-terminal polysiloxanes and monomeric silicon-containing species may also be used in the present invention.

The polyesters of the compositions of the invention preferably are prepared such that they have a molecular weight within the preferred range of 500 to 100,000 and a solubility level which provides sufficient compatibility and co-solubility with the active ingredients as described further below as well as co-solubility with the wide range of general additives and base formulations for topically applied compositions. Preferably, the polyesters of the compositions of the invention have a Hildebrand solubility parameter which is from about 5 to about 25, and more preferably from about 5 to 12. Hansen-type or other solubility parameter theories may also be used as guidelines in determining a useful polyester from the preferred polyesters of the present invention. Using a polyester having a solubility level which is similar to both the active ingredients and the other components within a given cosmetic formulation minimizes separation of the formulation into phases. Solubility parameters, such as the Hildebrand parameters, may be easily found either in published information or readily discernible experimentally by any manner suitable in the art. In selecting a preferred polyester for a given composition according to the present invention, the solubility parameters of the active ingredient and other components in the topical formulation are preferably taken into account.

In order to select the appropriate polyester having the same or a similar solubility parameter, the factors to be taken into account are the ester density, degree of cross-linking and chain branching, the presence and quantity of functional groups and molecular weight. Because ester linkages are polar, the higher the ester density, in general, the greater the solubility parameter. Further mild crosslinking and chain branching will tend to increase intermolecular distance which will tend to disrupt the polyester's ability to interact favorably with other like molecules which would tend to lower the solubility parameter. If the polyester becomes highly cross-linked, the solubility behavior changes and is not easily predicted by solubility parameters, such that for these particular polyesters, the solubility will have to be experimentally determined. Use of or increase in number of functional groups will generally increase the solubility parameter for the polyester due to the polarity of most functional groups. Increases in molecular weight, in the absence of cross-linking, will typically reduce the solubility parameter at least to a point where additional chain length will have little effect. At very high molecular weight, the solubility parameters are less accurate such that predicting the solubility behavior will also have to be determined experimentally.

In one embodiment, the polyester resin is a polyester having a backbone derived from the reaction of at least one linear or branched diol and at least one linear or branched diacid. The polyester resin has a molecular weight of from about 500 to about 100,000.

In another embodiment, the polyester resin is a saturated crosslinked hydroxy functional; polyester, comprised of glycerin, diethylene glycol, adipate crosslinked polymer, also known in its INCI designation as adipic acid/diethylene glycol/glycerin crosspolymer, sold as Lexorez® 100 by the Inolex Chemical Company, Philadelphia, Pa.

In a further embodiment, the polyester resin is comprised of hexanedioic acid, 1,2,3-propanediol, 2,2,4 trimethyl-1,3-pentanediol crosslinked polymer, also known in its INCI designation as trimethylpentanediol/adipic acid/glycerin crosspolymer, sold as Lexorez® 200 by the Inolex Chemical Company, Philadelphia, Pa.

While the structuring polymers and polyester film formers may be referred to in this detailed description in the singular form, it will be understood, based on this disclosure, that more than one polyester and/or polyheteroatom polymer may be used in compositions within the scope of the invention.

Additional Film Formers

The composition according to the invention may further contain at least one additional film former chosen from the list of film formers set forth on pages 2903-2906 of the CTFA International Cosmetic Ingredient Dictionary, 9^{th} edition (2002), the disclosure of which is specifically incorporated by reference herein.

The at least one additional film former which also may be used within the framework of the invention includes film formers having any film former chemistry known in the art such as: PVP, acrylates, and urethanes; synthetic polymers of the polycondensate type or free-radical type, or ionic type, polymers of natural origin and mixtures thereof or any other film former known within the practice of the cosmetic and pharmaceutical arts which one skilled in the art may determine to be compatible.

In one embodiment, the at least one additional film former is chosen from polyvinylpyrrolidones. Polyvinylpyrrolidones are available from, for example, ISP in different viscosity grades under the tradename PVP-K.

The at least one additional film former may also be chosen from, for example, polyethylene; vinylpyrrolidone/vinyl acetate (PVP/VA) copolymers such as the Luviskol® VA grades (all ranges) from BASF® Corporation and the PVP/VA series from ISP; acrylic fluorinated emulsion film formers including Foraperle® film formers such as Foraperle® 303 D from Elf Atochem (although Foraperle® may not be appropriate for some cosmetic formulations); GANEX® copolymers such as butylated PVP, PVP/Hexadecene copolymer, PVP/Eicosene copolymer or tricontanyl; Poly(vinylpyrrolidone/diethylaminoethyl methacrylate) or PVP/Dimethylaminoethylmethacrylate copolymers such as Copolymer 845; Resin ACO-5014 (Imidized IB/MA copolymer); other PVP based polymers and copolymers; alkyl cycloalkylacrylate copolymers (See WO 98/42298, the disclosure of which is hereby incorporated by reference); Mexomere® film formers and other allyl stearate/vinyl acetate copolymers (allyl stearate/VA copolymers); polyolprepolymers such as PPG-12/SMDI copolymer, polyolprepolymers such as PPG-1 2/SM D1 copolymer, Poly(oxy-1,2-ethanediyl), α-hydro-ω-hydroxy-polymer with 1,1′-methylene-bis-(4-isocyanatocyclohexane) available from Barnet; Avalure™ AC Polymers (Acrylates Copolymer) and Avalure™ UR polymers (Polyurethane Dispersions), available from BF Goodrich.

The at least one additional film former which also may be used within the framework of the invention includes film forming silicone resins. In one embodiment, the at least one film-forming silicone resin is chosen from silsesquioxanes and siloxysilicates. Any siloxysilicates or silsesquioxanes that function as a film-former are within the practice of the invention. In one embodiment, the at least one film-forming silicone resin is chosen from substituted siloxysilicates and silsesquioxanes. A substituted siloxysilicate or a substituted silsesquioxane may be, for example, a siloxysilicate or a silsesquioxane where a methyl group has been substituted with a longer carbon chain such as an ethane, propane, or butane. The carbon chain may be saturated or unsaturated.

In one embodiment, the additional film-forming silicone resin is chosen from siloxysilicates such as trimethylsiloxysilicates, which are represented by the following formula: [(CH₃)₃—Si—O]ₓ—(SiO_{4/2})_{y} (MQ Units) where x and y can have values ranging from 50 to 80. In a further embodiment, a siloxysilicate may be chosen from any combination of M and Q units, such as, for example, [(R)₃—Si—O]ₓ—(SiO_{4/2})_{y}, where R is chosen from a methyl group and longer carbon chains.

In a further embodiment, the film-forming silicone resin is chosen from silsesquioxanes that are represented by the following formula: (CH₃SiO_{3/2})ₓ (T Units) where x has a value of up to several thousand and the CH₃ may be replaced by an alkyl group. In one embodiment, the silsesquioxane is chosen from polymethylsilsesquioxanes, which are silsesquioxanes that do not have a substituent replacing the methyl group. The polymethylsilsesquioxanes useful in the present invention are film-formers and can, for example, have about 500 or less T units, such as from about 50 to about 500 T units. In another embodiment, they have a melting point from about 40° C. to about 80° C. These silicone resins are soluble or dispersible in volatile silicones or other organic liquids. Not all polymethylsilsesquioxanes are film-formers. For example, the highly polymerized polymethylsilsesquioxanes (T Resins), such as Tospearl™ from Toshiba or KMP590 from Shin-Etsu are highly insoluble and therefore are not effective film-formers. The molecular weight of these polymethylsilsesquioxanes is difficult to determine and they generally contain a thousand or more T units.

Other suitable polymethylsilsesquioxanes useful in accordance with the present invention include Belsil PMS MK, also referred to as Resin MK, available from Wacker Chemie. This polymethylsilsesquioxane is a polymer primarily formed of polymerized repeating units of CH₃SiO_{3/2} (T units) and which can also contain up to about 1% (by weight or by mole) of (CH₃)₂SiO_{2/2} (D units). The weight-average molecular weight can be, for example, from about 500 to about 50,000, such as about 10,000.

Other polymethylsilsesquioxanes suitable for use in the present invention also include KR-220L, KR-242A and KR-521 available from SHIN-ETSU.

In a further embodiment, the additional film-forming silicone resin may be chosen from combinations of M, D, T, and Q units comprising at least two units chosen from M, D, T, and Q and that satisfy the relationship RₙSiO_{(4-n)/2} wherein n is a value ranging from 1.0 to 1.50. Some resins of this type are disclosed in U.S. Pat. No. 6,074,654, the disclosure of which is incorporated by reference herein. R may be a methyl group or any carbon chain as long as the silicone resin retains its film forming properties. (Up to 5%, of silanol or alkoxy functionality may also be present in the resin structure as a result of processing.) The additional film-forming silicone resins may be solid at about 25° C. and may have a molecular weight ranging from 1000 to 10000 grams/mole.

In a further embodiment, the at least one film-forming silicone resin comprises repeating M units and Q units. The ratio of M units to Q units may be, for example, 0.7:1. The at least one film-forming silicone resin may be chosen from Wacker 803 and 804 available from Wacker Silicones Corporation and G.E. 1170-002 from General Electric.

In a further embodiment, the additional one film-forming silicone resin is a copolymer wherein at least one unit of the copolymer is chosen from M, D, T, and Q silicone units and at least one additional unit of the copolymer is chosen from an ester. The at least one film-forming silicone resin may be chosen from, for example, diisostearoyl trimethylolpropane siloxysilicates, such as SF 1318, available from GE Silicones.

Block Copolymer Film Formers

The at least one additional film former may be a block copolymer film former. This block copolymer film former may be chosen from di-block, tri-block copolymer film formers, multi-block copolymer film formers and radial block copolymer film formers. A block copolymer film former generally contains at least two thermodynamically incompatible segments. For example, a tri-block is usually defined as an A-B-A type copolymer or a copolymer having soft and hard segments in a ratio of one hard, one soft, and one hard segment. A multiblock, radial or star copolymer film former usually contains any combination of hard and soft segments, provided that there are both hard and soft characteristics. An example of a hard block copolymer segment is styrene, while examples of soft block copolymer segments include ethylene, propylene, and butylene or combinations thereof. Specific examples of block copolymer film formers include Kraton® rubbers available from the Shell Chemical company, gels such as Versagel MD 870, Versagel M5960 or Versagel M5970, which are available from Penreco of Houston Tex., and block copolymers available from Brooks Industries, such as Gel Base.

The at least one additional film former which also may be used within the framework of the invention includes film formers having any film former chemistry known in the art such as: PVP, acrylates, and urethanes; synthetic polymers of the polycondensate type or free-radical type, or ionic type, polymers of natural origin and mixtures thereof or any other film former known within the practice of the cosmetic and pharmaceutical arts which one skilled in the art may determine to be compatible.

An appropriate concentration of the at least one additional film former may be determined by one of skill in the art and can vary considerably based on the application. For example, for cosmetic compositions, at least one additional film former may be used in an amount from 0.1% to 20% such as, for example, from 1% to 10% by weight, relative to the total weight of the composition.

Pigments

Further, the composition of the present invention may also comprise at least one coloring agent. The at least one coloring agent may be chosen from pigments, dyes, such as liposoluble dyes, nacreous pigments, and pearling agents.

Representative liposoluble dyes which may be used according to the present invention include Sudan Red, DC Red 17, DC Green 6, B-carotene, soybean oil, Sudan Brown, DC Yellow 11, DC Violet 2, DC Orange 5, annatto, and quinoline yellow. The liposoluble dyes, when present, generally have a concentration ranging up to 20% by weight of the total weight of the composition, such as from 0.0001% to 6%.

The nacreous pigments which may be used according to the present invention may be chosen from white nacreous pigments such as mica coated with titanium or with bismuth oxychloride, colored nacreous pigments such as titanium mica with iron oxides, titanium mica with ferric blue or chromium oxide, titanium mica with an organic pigment chosen from those mentioned above, and nacreous pigments based on bismuth oxychloride. The nacreous pigments, if present, be present in the composition in a concentration ranging up to 50% by weight of the total weight of the composition, such as from 0.1% to 20%.

The pigments which may be used according to the present invention may be chosen from white, colored, inorganic, organic, polymeric, nonpolymeric, coated and uncoated pigments. Representative examples of mineral pigments include titanium dioxide, optionally surface-treated, zirconium oxide, zinc oxide, cerium oxide, iron oxides, chromium oxides, manganese violet, ultramarine blue, chromium hydrate, and ferric blue. Representative examples of organic pigments include carbon black, pigments of D & C type, and lakes based on cochineal carmine, barium, strontium, calcium, and aluminum.

If present, the pigments may be present in the composition in a concentration ranging up to 40% by weight of the total weight of the composition, such as from 1% to 35%, and further such as from 2% to 25%. In the case of certain products, the pigments, including nacreous pigments, may, for example, represent up to 50% by weight of the composition.

Waxes

For the purposes of the invention, the waxes are those generally used in cosmetics and dermatology; they are, for example, of natural origin, for instance beeswax, carnauba wax, candelilla wax, ouricury wax, Japan wax, cork fibre wax, sugar cane wax, paraffin wax, lignite wax, microcrystalline waxes, lanolin wax, montan wax, ozokerites and hydrogenated oils such as hydrogenated jojoba oil as well as waxes of synthetic origin, for instance polyethylene waxes derived from the polymerization of ethylene, waxes obtained by Fischer-Tropsch synthesis, fatty acid esters and glycerides that are solid at 40° C., for example, at above 55° C., silicone waxes such as alkyl- and alkoxy-poly(di)methylsiloxanes and/or poly(di)methyl-siloxane esters that are solid at 40° C., for example, at above 55° C.

Further, the composition of the present invention may also comprise at least one thickener, humectant, and/or emulsifier. Suitable thickeners, humectants, and emulsifiers can be chosen by the skilled artisan and include, but are not limited to, those disclosed in “Encyclopedia of Polymers and Thickeners for Cosmetics,” R. Y. Lochhead and W. R. Fron, eds., Cosmetics & Toiletries, vol. 108, pp. 95-135 (May 1993); and International Cosmetic Ingredient Dictionary and Handbook (7^{th} Ed.) vol. 1-3 (1997), The Cosmetic, Toiletry and Fragrance Association, Washington, D.C, both of which are incorporated herein by reference.

EXAMPLE 1
Mascara composition

| Phase | INCI Name | % |
|---|---|---|
| A | DI Water | 44.53 |
| | Thickener | 0.20 |
| | Film formers | 1.00 |
| | Humectant | 2.00 |
| | Preservatives | 0.45 |
| | Triethanolamine | 1.67 |
| B | Fatty Phase (Waxes and Oils) | 11.85 |
| | Emulsifier | 3.75 |
| | Stearic Acid | 3.00 |
| | Preservative | 0.05 |
| | Polyamide Resin (Uniclcar 100) | 8.50 |
| | Film Former | 3.00 |
| | Polyester (Lexorez 200) | 5.00 |
| | Pigments | 5.00 |
| C | Antifoam | 0.10 |
| D | Latex film former | 10.00 |
| E | Preservatives | 1.10 |
| | Polysilicone 11 Film former | |

The composition exhibits good wear.

In main vessel, combine all of phase B and homogenize for 1 hour while maintaining the temp. 82-87° C.

In a separate vessel, combine phase A and begin heating to 85° C.

While maintaining the temperature, add phase A to phase B.

Add phase C to main batch.

Homogenize for 20 minutes at 82-87° C.

Begin to cool batch to 70° C. and switch to a sweep blade.

At 60° C., add phase D.

At 40° C., add phase.

Drop batch at 30° C.

EXAMPLE 2
Mascara composition

| Phase | INCI Name | % w/w |
|---|---|---|
| A | Volatile Solvent | 31.19 |
| | Silicone Film former | 8.00 |
| | Versagel MD 870 | 15.00 |
| | quaternium-18 hectorite | 5.50 |
| | Pigments | 6.00 |
| B | Propylene Carbonate | 1.80 |
| C | Waxes | 16.00 |
| | Polyamide Resin (Uniclear 100) | 6.00 |
| | Polyester Lexorez 200 | 3.00 |
| | Preservative | 0.01 |
| | Film Former | 2.00 |
| | Emulsifier | 1.00 |
| D | Latex film former | 5.00 |

The composition exhibits good wear.

Processing Instructions:

In the main beaker, weigh out phase A and mix on the homogenizer for 40 minutes at room temperature. Once the batch has been mixing for 40 minutes, slowly add half of phase B to the main beaker. Begin heating to 70° C. In a separate beaker combine phase C and heat to 85-90° C. with propeller mixer. Once phase A and phase C have reached their optimal temperatures, add phase C to phase A. Allow batch to homogenize for 5 minutes while maintaining heat at 80-85° C. Add the remainder of phase B to main beaker and continue homogenizing for 30 minutes while maintaining temperature at 80-85° C. After previous step is completed, remove batch from the homogenizer and begin cooling to 30-35° C. using sweep mixing.

EXAMPLE 3
Lipstick composition

| Phase | Ingredients | % w/w |
|---|---|---|
| A | Fatty phase (Emollients) | 67.91 |
| | Preservative | 0.05 |
| | Polyester film former (Lexorez 200) | 6.00 |
| B | Polyamide resin (Uniclear 100) | 3.00 |
| | Waxes | 15.20 |
| C | Pigments | 2.34 |
| D | Fillers | 5.50 |

This composition exhibits good wear.

Manufacturing Procedure

Combine all the ingredients of Phase A into the Mixing Kettle. Heat to 85° C.-95° C. under medium agitation and mix for 20-30 minutes. Charge a portion of the emollients oil phase (Phase A) into the Disconti Mill. Heat to about 65° C. Add the pigments and fillers (Phase C). Mill for 40-45 min. at 60-65° C. Melt the waxes (Phase B) at 100-105° C. in the melting kettle. Discharge the color phase (Phase C) from the mill. Rinse the mill with the remaining oil phase for 20-30 min. Complete the color phase with the rinse residual. Add the color phase into the melting kettle. Heat to 100° C.-105°. Mix for 20-30 minutes. Add Phase D. Mix 20-30 minutes or until homogeneous. Lower the temperature of the batch between 82° C.-85° C. Mix 10-20 minutes or until homogeneous. Pour the batch into a mold.

1. A composition comprising
a liquid fatty phase, which comprises at least one structuring polymer having a polymer skeleton with at least one hydrocarbon-based repeating unit, and at least one heteroatom; and
at least one film-forming polyester.

2. The composition of claim 1, wherein the film-forming polyester is present in an amount effective to provide transfer resistant properties.

3. The composition of claim 1, wherein the liquid fatty phase further comprises at least one of a polar oil and an apolar oil.

4. The composition of claim 1, wherein the liquid fatty phase further comprises at least one of a non-volatile oil and volatile oil.

5. The composition of claim 1, wherein the liquid fatty phase further comprises a dispersion of lipid vesicles.

6. The composition of claim 1, wherein the structuring polymer is a solid that is not deformable at 25° C. and 700 mmHg.

7. The composition of claim 1, wherein the structuring polymer further comprises at least one terminal fatty alkyl or alkenyl chain bonded to the polymer skeleton via at least one linking group.

8. The composition of claim 1, wherein the at least one structuring polymer further comprises at least one pendant fatty alkyl or alkenyl chain bonded to any carbon or hetero atom of the polymer skeleton via at least one linking group.

9. The composition of claim 8, wherein the pendant fatty alkyl or alkenyl chain is functionalized.

10. The composition of claim 1, wherein the at least one hydrocarbon-based repeating unit and at least one hetero atom is selected from the group consisting of an amide, a carbamate, and a urea.

11. The composition of claim 10, wherein the liquid fatty phase comprises at least one polar oil.

12. The composition of claim 1, wherein the at least one structuring polymer is a polyamide comprising a polymer skeleton with at least one amide repeating unit.

13. The composition of claim 12, wherein the structuring polymer further comprises at least one pendant fatty chain and/or at least one terminal chain comprising from 8 to 20 carbon atoms bonded to at least one of the amide repeating units via at least one linking group.

14. The composition of claim 1, wherein the at least one structuring polymer is capable of structuring the composition without opacifying the composition.

15. The composition of claim 1, wherein the at least one structuring polymer is a compound of formula (I): 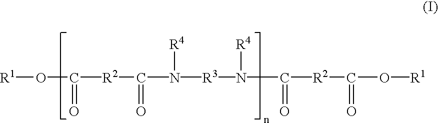
wherein n is an integer which represents the number of amide units such that the number of ester groups present in said at least one polyamide polymer ranges from 10% to 50% of the total number of all said ester groups and all of said amide groups comprised in said at least one polyamide polymer;
R¹ are independently alkyl groups comprising at least 4 carbon atoms, or alkenyl groups comprising at least 4 carbon atoms;
R² are independently C₄ to C₄₂ hydrocarbon-based groups with the proviso that at least 50% of all R² are chosen from C₃₀ to C₄₂ hydrocarbon-based groups;
R³ is an organic group comprising atoms selected from the group consisting of carbon atoms, hydrogen atoms, oxygen atoms and nitrogen atoms with the proviso that R³ comprises at least 2 carbon atoms; and
R⁴ are independently hydrogen atoms, C₁ to C₁₀ alkyl groups, and a direct bond to at least one of R³ and another R⁴ such that when said at least one group another R⁴, the nitrogen atom to which both R³ and R⁴ are bonded forms part of a heterocyclic structure defined in part by R⁴—N—R³, with the proviso that at least 50% of all R⁴ are hydrogen atoms.

16. The composition of claim 1, wherein the at least one film-forming polyester comprises a backbone obtainable by reacting at least one diol and at least one diacid.

17. The composition of claim 1, wherein the at least one film-forming polyester comprises the compound of formula (II): 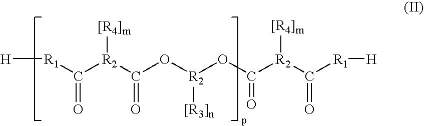
wherein:
R₁ is independently either —O— or —O—R₂—O—;
R₂ is independently a linear or branched C₂-C₆₀ aliphatic group;
R₃ is —O—(C═O)—R₁—H, or —O—[(C═O)—R₂(R₄)ₘ—(C═O)—O—R₂(R₃)ₙ—O]ₚ(C═O)—R₂(R₄)ₘ—(C═O)—R₁—H;
R₄ is —(C═O)—O—R₁—H, H, or —(C═O)—O—R₂—O—[(C═O)—R₂(R₄)ₘ+C═O)—O—R₂(R₃)ₙ—O]ₚHC═O)—R₂(R₄)ₘ—(C═O)—R₁—H;
m is independently an integer and 1<=m<=5;
n is independently an integer and 1<=n<=5; and
p is independently an integer and 3<=p<=2000.

18. The composition of claim 1, wherein the at least one film-forming polyester is a compound of formula (III): 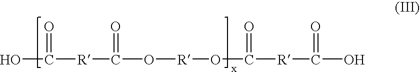
wherein R′ are independently a linear hydroxyl-substituted aliphatic C₂-C₆₀ group with from 0 to 200 ether linkages, a branched hydroxy-substituted aliphatic C₂-C₆₀ group with from 0 to 200 ether linkages, an linear unsubstituted aliphatic C₂-C₆₀ group with from 0 to 200 ether linkages, branched unsubstituted aliphatic C₂-C₆₀ group with from 0 to 200 ether linkages, a hydroxy-substituted cyclic aliphatic C₂-C₁₂ group, or an unsubstituted cyclic aliphatic C₂-C₁₂ group, a hydroxy-substituted aromatic group, or an unsubstituted cyclic aliphatic C₂-C₁₂ group; and
x is independently an integer and 3<=x<=2000.

19. The composition of claim 1, wherein the at least one film-forming polyester is a compound of formula (IV): 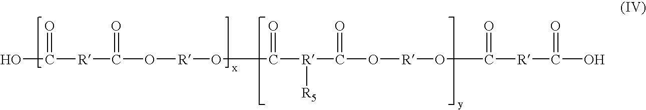
wherein R′ are independently a linear hydroxyl-substituted aliphatic C₂-C₆₀ group with from 0 to 200 ether linkages, a branched hydroxy-substituted aliphatic C₂-C₆₀ group with from 0 to 200 ether linkages, an linear unsubstituted aliphatic C₂-C₆₀ group with from 0 to 200 ether linkages, branched unsubstituted aliphatic C₂-C₆₀ group with from 0 to 200 ether linkages, a hydroxy-substituted cyclic aliphatic C₂-C₁₂ group, or an unsubstituted cyclic aliphatic C₂-C₁₂ group, a hydroxy-substituted aromatic group, or an unsubstituted cyclic aliphatic C₂-C₁₂ group;
R₅ is —H, —(C═O)—OH or —(C═O)—R′—R₅)_{q}—(C═O)—O—R′—O]_{y}—(C═O)—R′(R₅)_{q}—(C═O)—OH;
x is independently an integer and 3<=x<=2000,
y is independently an integer and 3<=y<=2000; and
q is independently an integer and 0<=q<=5.

20. The composition of claim 1, wherein the at least one film-forming polyester is a compound of formula (V): 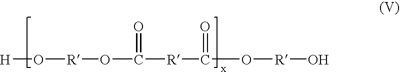
wherein R′ are independently a linear hydroxyl-substituted aliphatic C₂-C₆₀ group with from 0 to 200 ether linkages, a branched hydroxy-substituted aliphatic C₂-C₆₀ group with from 0 to 200 ether linkages, an linear unsubstituted aliphatic C₂-C₆₀ group with from 0 to 200 ether linkages, branched unsubstituted aliphatic C₂-C₆₀ group with from 0 to 200 ether linkages, a hydroxy-substituted cyclic aliphatic C₂-C₁₂ group, or an unsubstituted cyclic aliphatic C₂-C₁₂ group, a hydroxy-substituted aromatic group, or an unsubstituted cyclic aliphatic C₂-C₁₂ group; and
x is independently an integer and 3<=x<=2000.

21. The composition of claim 1, wherein the at least one film-forming polyester is a compound of formula (VI): 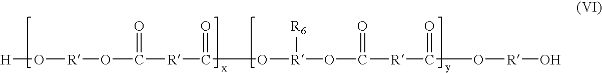
wherein R′ are independently a linear hydroxyl-substituted aliphatic C₂-C₆₀ group with from 0 to 200 ether linkages, a branched hydroxy-substituted aliphatic C₂-C₆₀ group with from 0 to 200 ether linkages, an linear unsubstituted aliphatic C₂-C₆₀ group with from 0 to 200 ether linkages, branched unsubstituted aliphatic C₂-C₆₀ group with from 0 to 200 ether linkages, a hydroxy-substituted cyclic aliphatic C₂-C₁₂ group, or an unsubstituted cyclic aliphatic C₂-C₁₂ group, a hydroxy-substituted aromatic group, or an unsubstituted cyclic aliphatic C₂-C₁₂ group;
R₆ is —H, OH or —[O—R′(R₆)_{q}—O—(C═O)—R′—(C═O)]_{y}O—R′(R₆)_{q}—OH;
x is independently an integer and 3<=x<=2000;
y is independently an integer and 3<=y<=2000; and
q is independently an integer and 0<=q<=5.

22. The composition of claim 1, wherein the at least one film-forming polyester is a compound of formula (VII): 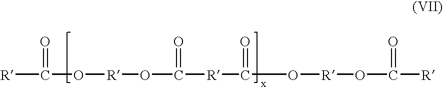
wherein R₆ is —H, OH or —[O—R′(R₆)_{q}—O—(C═O)—R′—(C═O)]_{y}—O—R′(R₆)_{q}—OH.; and
x is independently an integer and 3<=x<=2000.

23. The composition of claim 1, wherein the at least one film-forming polyester is a compound of formula (VIII): 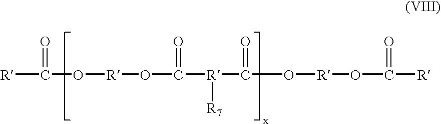
wherein R′ are independently a linear hydroxyl-substituted aliphatic C₂-C₆₀ group with from 0 to 200 ether linkages, a branched hydroxy-substituted aliphatic C₂-C₆₀ group with from 0 to 200 ether linkages, an linear unsubstituted aliphatic C₂-C₆₀ group with from 0 to 200 ether linkages, branched unsubstituted aliphatic C₂-C₆₀ group with from 0 to 200 ether linkages, a hydroxy-substituted cyclic aliphatic C₂-C₁₂ group, or an unsubstituted cyclic aliphatic C₂-C₁₂ group, a hydroxy-substituted aromatic group, or an unsubstituted cyclic aliphatic C₂-C₁₂ group;
R₇ is H, —(C═O)—OH or —[O—R′—O—(C═O)—R′(R₇)_{q}—(C═O)]ₓ, —R′—O—C═O)]ₓ—O—R′—O—(C═O)—R′(R₇)_{q};
x is independently an integer and 3<=x<=2000; and
q is independently an integer and 0<=q<=5.

24. The composition of claim 1, wherein the at least one film-forming polyester is a compound of formula (IX): 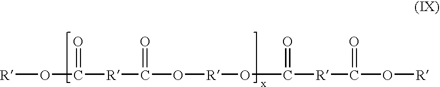
wherein R′ are independently a linear hydroxyl-substituted aliphatic C₂-C₆₀ group with from 0 to 200 ether linkages, a branched hydroxy-substituted aliphatic C₂-C₆₀ group with from 0 to 200 ether linkages, an linear unsubstituted aliphatic C₂-C₆₀ group with from 0 to 200 ether linkages, branched unsubstituted aliphatic C₂-C₆₀ group with from 0 to 200 ether linkages, a hydroxy-substituted cyclic aliphatic C₂-C₁₂ group, or an unsubstituted cyclic aliphatic C₂-C₁₂ group, a hydroxy-substituted aromatic group, or an unsubstituted cyclic aliphatic C₂-C₁₂ group; and
x is independently an integer and 3<=x<=2000.

25. The composition of claim 1, wherein the at least one film-forming polyester is a compound of formula (X): 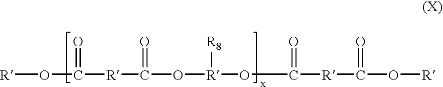
wherein R′ are independently a linear hydroxyl-substituted aliphatic C₂-C₆₀ group with from 0 to 200 ether linkages, a branched hydroxy-substituted aliphatic C₂-C₆₀ group with from 0 to 200 ether linkages, an linear unsubstituted aliphatic C₂-C₆₀ group with from 0 to 200 ether linkages, branched unsubstituted aliphatic C₂-C₆₀ group with from 0 to 200 ether linkages, a hydroxy-substituted cyclic aliphatic C₂-C₁₂ group, or an unsubstituted cyclic aliphatic C₂-C₁₂ group, a hydroxy-substituted aromatic group, or an unsubstituted cyclic aliphatic C₂-C₁₂ group;
R₈ is H, —OH, or —[(C═O)—R′—(C═O)—O—R′(R₈)_{q}—O—]ₓ—(C═O)—R′—(C═O)—O—R′(R₈); and
x is independently an integer and 3<=x<=2000.

26. The composition of claim 1, further comprising at least one additional film-former.

27. The composition of claim 1, further comprising at least one pigment.

28. The composition of claim 1, further comprising a wax.

29. The composition of claim 1, further comprising at least one thickner, humectant or emulsifier.

30. A method of making up the skin and/or hair comprising applying the composition of claim 1 to the skin and/or hair.
